(19) United States
(12) Patent Application Publication
(10) Pub. No.: US 2026/0201414 A1
(43) Pub. Date: Jul. 16, 2026

(54) METHOD FOR PREPARING CORONA VIRUS-LIKE PARTICLES AND VACCINE COMPRISING THE SAME

(71) Applicant: National Defense Medical University, Taipei City (TW)

(72) Inventors: SZU-CHENG KUO, Taoyuan City (TW); HUI-TSU LIN, New Taipei City (TW); DER-JIANG CHIAO, Kaohsiung City (TW); CHIN-MAO HUNG, Taoyuan City (TW); YUNG-CHIH SUN, Taipei City (TW); TIEN-YAO CHANG, Taipei City (TW)

(21) Appl. No.: 19/353,267

(22) Filed: Oct. 8, 2025

(30) Foreign Application Priority Data
Jan. 13, 2025 (TW) 114101337

(51) Int. Cl. C12N 15/86; A61K 39/215; A61P 37/04

(52) U.S. Cl. CPC C12N 15/86; A61K 39/215; A61P 37/04; C12N 2710/14043; C12N 2770/20022; C12N 2770/20023; C12N 2770/20034; C12N 2770/20052

(57) ABSTRACT

The present invention provides a method for producing coronavirus virus-like particles (VLPs) and a vaccine comprising the same. The method comprises: constructing a recombinant baculovirus carrying a coronavirus spike protein gene; transducing mosquito cells with the recombinant virus; and culturing the transduced cells in a serum-free medium to produce VLPs containing the coronavirus spike protein. Preferably, the mosquito cells are C6/36 cells, and the spike protein gene may be derived from SARS-COV, MERS-COV, or SARS-COV-2. Furthermore, the invention provides a vaccine containing the VLPs produced by the above method, which can be administered intranasally or by intramuscular injection.

### CROSS-REFERENCE TO RELATED APPLICATIONS

This application claims priority to Taiwan Invention patent application No. 114101337, filed on Jan. 13, 2025, the entire disclosure of which is hereby incorporated by reference.

### BACKGROUND OF THE INVENTION

### 1. Field of the Invention

The present invention pertains to the fields of virology and vaccinology, and more particularly relates to a method for producing coronavirus virus-like particles (VLPs) and the applications thereof. Specifically, the present invention provides a novel method that employs a recombinant baculovirus system and a serum-free culture medium to produce coronavirus VLPs. The resulting adjuvant-free vaccine can be effectively administered either via intranasal instillation or intramuscular injection to elicit an immune response.

### 2. Description of the Prior Art

The COVID-19 pandemic has had a profound impact on global public health and economic systems. Although multiple vaccines have been developed, there remains room for improvement, particularly in enhancing mucosal immunity and addressing viral mutations.

Conventional vaccines are primarily administered via intramuscular injection; however, many pathogens—including Severe Acute Respiratory Syndrome Coronavirus 2 (SARS-COV-2)—enter the human body through mucosal surfaces. Accordingly, the development of mucosal vaccines capable of eliciting secretory antibodies in the respiratory tract is critical for effective control of SARS-COV-2 and its variants.

Virus-like particles (VLPs), owing to their favorable antigenicity and high safety profile, have long been regarded as promising candidates in vaccine development. For instance, NVX-CoV2373—an intramuscular nanoparticle-based vaccine developed by Novavax, USA—targets SARS-COV-2 and has demonstrated good safety and high efficacy in clinical trials.

Intranasal administration has emerged as an attractive route for mucosal vaccine delivery, potentially offering superior sterilizing immunity against mucosal pathogens compared to intramuscular injection. Nevertheless, the development of intranasal vaccines faces several challenges, including the mucus and epithelial barriers, as well as the lack of immunoadjuvants compatible with the nasal environment.

In conventional method, the production of coronavirus virus-like particles (VLPs) typically requires co-expression of multiple structural proteins in mammalian or insect cells, which increases the complexity and cost of manufacturing. Furthermore, many existing VLP production systems utilize serum-containing culture media, potentially introducing additional variability and safety concerns.

In view of the limitations in current coronavirus vaccine development—particularly in inducing mucosal immunity, addressing viral mutations, and streamlining production—the inventors of the present invention initiated a comprehensive improvement effort. Existing VLP production methods typically require co-expression of multiple structural proteins in mammalian or insect cells, resulting in complex manufacturing processes and increased costs. In addition, the use of serum-containing media introduces batch variability and potential safety risks. Although intranasal vaccines show promise in mucosal immunization, their development is hindered by biological barriers such as mucus and epithelium, as well as the lack of compatible immunoadjuvants. To address these challenges, the present invention provides a simplified method for VLP production, employing a serum-free culture medium and the baculovirus/mosquito (BacMos) expression system expressing a single structural protein. This approach reduces manufacturing complexity while improving product consistency and safety. Moreover, the adjuvant-free intranasal vaccine formulation developed herein demonstrates the potential to elicit robust mucosal immunity, offering a novel strategy for the prevention of SARS-COV-2 infection and its variants.

### SUMMARY OF THE INVENTION

The primary objective of the present invention is to provide a method for producing coronavirus virus-like particles, comprising the following steps:
a) providing a recombinant baculovirus carrying a coronavirus spike(S) protein gene;
b) transducing an insect cell with the recombinant baculovirus from step (a); and
c) culturing the insect cell transduced in step (b) in a serum-free medium to produce a coronavirus virus-like particle;
wherein the coronavirus virus-like particle comprises a coronavirus spike(S) protein.

In the aforementioned method, the insect cell is a mosquito-derived cell, specifically selected from either C6/36 or AP-61 cell lines.

Additionally, in the aforementioned method, the coronavirus spike protein gene may be derived from Severe Acute Respiratory Syndrome Coronavirus (SARS-COV), Middle East Respiratory Syndrome Coronavirus (MERS-COV), or Severe Acute Respiratory Syndrome Coronavirus 2 (SARS-COV-2).

Furthermore, in the aforementioned method, the coronavirus spike protein gene is a prefusion-stabilized full-length spike gene, which includes a mutation at the furin cleavage site and a 2P mutation (two proline substitutions).

Furthermore, in the aforementioned method, the recombinant baculovirus further comprises the following components:
an hr1 pag1 mosquito promoter;
a Japanese encephalitis virus prM signal peptide;
a Rhopalosiphum padi virus 5′-untranslated region internal ribosome entry site (RhPV 5′-UTR IRES; abbreviated as Rhir);
an enhanced green fluorescent protein (eGFP) gene; and
a translation stop codon.

In one embodiment, the serum-free culture medium used in the above method comprises the following components:
RPMI-1640 medium;
tryptose phosphate broth (TPB) at 0.3%;
Pluronic® F-68 at 0.2%;
peptone primatone at 0.5%;
yeastolate at 0.4%; and
lipid mixture at 0.1%.

Additionally, the present invention provides a vaccine formulation comprising the coronavirus virus-like particles produced by the aforementioned method. The vaccine may be administered intranasally or via intramuscular injection, and is capable of eliciting robust mucosal and systemic immune responses without the need for an adjuvant. This adjuvant-free approach not only simplifies the production process but also reduces the risk of side effects associated with adjuvants. Notably, when administered intranasally, the vaccine can directly activate the mucosal immune system in the respiratory tract, serving as a frontline defense to block viral transmission and proliferation during the early stages of infection. Furthermore, due to the use of a serum-free medium in VLP production, the resulting vaccine offers enhanced safety and batch-to-batch consistency. This novel vaccine holds significant promise in the prevention and control of SARS-COV-2 and its variants, providing a valuable tool for global public health efforts.

### BRIEF DESCRIPTION OF THE DRAWINGS

FIG. 1 illustrates the basic structure of the BacMos system and the production process of coronavirus virus-like particles (VLPs);

FIG. 2A shows immunofluorescence staining of SARS-COV-2 spike protein in transduced cells;

FIG. 2B demonstrates the secretion of a full-length spike protein;

FIG. 2C presents the secretion of wild-type and 2P mutant MERS-COV spike proteins in transduced cells;

FIG. 2D shows the expression of 2P-mutant SARS-COV (2003) spike protein in transduced cells;

FIG. 3A shows the sucrose density gradient centrifugation result of SARS-CoV-2 VLPs;

FIG. 3B presents the electron microscopy image of SARS-COV-2 VLPs;

FIG. 4A shows the binding affinity of SARS-COV-2 VLPs to different receptors;

FIG. 4B illustrates the inhibition assay of SARS-COV-2 VLPs binding to human ACE2;

FIG. 5A depicts the mouse immunization schedule;

FIG. 5B-5D show the responses of total IgG, IgG1, and IgG2a specific to SARS-COV-2 S protein after the second and third immunizations;

FIG. 6A demonstrates the systemic SARS-COV-2 S-specific IgA response;

FIG. 6B shows the secretory IgA response specific to SARS-COV-2 S protein in bronchoalveolar lavage fluid and intestinal mucosa;

FIG. 6C presents the total IgG mucosal response specific to SARS-COV-2 S protein in bronchoalveolar lavage fluid;

FIG. 7A shows the systemic neutralizing antibody response against the wild-type virus strain;

FIG. 7B demonstrates the systemic neutralizing antibody response against viral variants;

FIG. 7C shows the mucosal neutralizing antibody responses in bronchoalveolar lavage fluid against the wild-type and Omicron variants;

FIG. 8A-8B show the duration of systemic immune responses induced by intranasal or intramuscular immunization with SARS-COV-2 VLPs;

FIG. 9A-9C demonstrate the protective efficacy of intranasal immunization with SARS-COV-2 VLPs in K18-hACE2 transgenic mice.

FIG. 10 presents the immunofluorescence analysis of MERS-COV VLPs expression;

FIG. 11 shows the Western blot analysis of MERS-COV spike protein secretion;

FIG. 12A presents the tangential flow filtration (TFF) purification and Western blot analysis of MERS-COV VLPs;

FIG. 12B shows the sucrose gradient purification and dot blot analysis of MERS-COV VLPs;

FIG. 13 demonstrates the expression and immunofluorescence staining analysis of SARS-COV spike protein in AP-61 cells;

FIG. 14 shows the Western blot analysis of spike protein secretion in transduced mosquito cells;

FIG. 15 presents the optimization of SARS-COV-2 VLPs production under different cell lines, culture conditions, and MOIs;

FIG. 16 shows the optimization analysis of MERS-COV VLPs production in C6/36 cells under serum-free medium (SFM) conditions;

FIG. 17 presents the optimization analysis of SARS-COV VLPs production in C6/36 cells under serum-free medium (SFM) conditions;

FIG. 18A illustrates the experimental design for evaluating the immunogenicity and protective efficacy of SARS-COV-2 VLPs in the hamster model; and

FIG. 18B shows the comparison of viral RNA levels in hamster lung tissues after vaccination with SARS-COV-2 VLPs.

### DETAILED DESCRIPTION OF THE PREFERRED EMBODIMENTS

All technical and scientific terms used in this specification, unless otherwise defined, shall have meanings commonly understood by those skilled in the art. The following examples are provided to illustrate the present invention and are not intended to limit its scope in any way. Unless otherwise specified, the materials used herein are commercially available, and the sources listed below are provided by way of example only.

### Example 1—Construction of Recombinant Baculoviruses

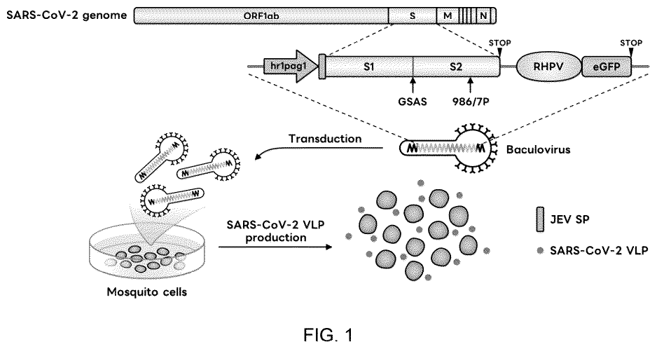

Referring to FIG. 1, in this example, a recombinant baculovirus carrying the spike protein gene of MERS-COV, SARS-COV, or SARS-COV-2 was constructed using the baculovirus/mosquito (BacMos) expression system. The spike protein gene is in a prefusion-stabilized form, incorporating a furin cleavage site mutation and a 2P (double proline) mutation. The recombinant virus also contains an hr1 pag1 mosquito-specific promoter, a Japanese encephalitis virus (JEV) prM signal peptide, a RhPV 5′-UTR IRES element, an enhanced green fluorescent protein (eGFP) gene, and a translation stop codon.

### 1. Gene Construction

In this step, the spike protein gene of MERS-COV, SARS-COV, or SARS-COV-2 was inserted into a baculovirus vector using the BacMos system. The gene was modified to include a furin cleavage site mutation (GSAS) and a 2P mutation (proline substitutions at positions 986 and 987) to ensure stability in the prefusion conformation. The recombinant baculovirus was constructed with the following components:
hr1 pag1: a mosquito-specific promoter for initiating transcription of the target gene;
JEV SP: Japanese encephalitis virus prM signal peptide to facilitate spike protein expression;
S1-S2: a prefusion-stabilized full-length spike gene containing the furin cleavage site mutation and 2P mutation;
Rhir: Rhopalosiphum padi virus (RhPV) 5′-untranslated region internal ribosome entry site (5′-UTR IRES);
eGFP: enhanced green fluorescent protein gene;
▾: translation stop codon.

As shown in FIG. 1, the diagram illustrates the basic structure of the BacMos system and the production process of SARS-COV-2 virus-like particles (VLPs). The recombinant baculovirus (BacMos-monomeric S), which contains the aforementioned DNA sequence, can efficiently transduce mosquito cells and induce the expression and secretion of the SARS-COV-2 spike protein.

### 2. Virus Preparation and Transduction

In this step, the recombinant baculovirus was used to express proteins in insect cells (C6/36 cell line, Aedes albopictus) via the BacMos system. Notably, in the present invention, the insect cells were cultured in a serum-free medium, which represents a key innovation of the invention. A detailed comparison of the serum-free medium versus the conventional serum-containing medium is summarized below in Table 1:

TABLE 1
Comparison between serum-free and serum-containing media

| Characteristics | Serum-free medium | Transitional cell medium |
|---|---|---|
| Components | Basal medium: RPMI-1640; | Basal medium: RPMI-1640; |
| | 0.3% tryptose phosphate broth | 10% serum (fetal bovine serum |
| | (TPB); | or bovine growth serum); |
| | 0.2% Pluronic ® F-68; | Growth factors, etc. |
| | 0.5% peptone primatone; | |
| | 0.4% yeastolate; | |
| | 0.1% lipid mixture. | |
| Advantages | 1. Safety: Animal serum may | 1. Generally compatible with |
| | carry unknown pathogens; | the growth of most cell lines; |
| | serum-free culture eliminates | 2. Easier to establish optimized |
| | such potential risks. | production conditions. |
| | 2. Product consistency and | |
| | controllability: Eliminates | |
| | batch-to-batch variability in | |
| | serum, improving the | |
| | reproducibility of VLP | |
| | production. | |
| | 3. Simplified purification | |
| | process: Serum contains a | |
| | large number of proteins and | |
| | other components that | |
| | require additional removal | |
| | steps. | |
| | 4. Lower production cost: | |
| | Serum is expensive, whereas | |
| | most components of | |
| | serum-free media can be | |
| | sterilized by | |
| | high-temperature | |
| | autoclaving. | |
| | 5. Avoidance of batch | |
| | variability: Further | |
| | enhances the | |
| | reproducibility of VLP | |
| | production. | |
| | 6. Environmental and ethical | |
| | benefits: Reduces reliance | |
| | on animal-derived | |
| | materials, addressing | |
| | societal concerns over | |
| | animal welfare and ethics. | |
| Disadvantages | Different cell lines require | 1. High cost, which increases |
| | distinct serum-free culture | the production cost of |
| | conditions, necessitating | biopharmaceutical products; |
| | extensive trial and error. | 2. Potential contamination risk |
| | | from serum-derived sources; |
| | | 3. Complex composition, which |
| | | may act as allergens; |
| | | 4. Batch-to-batch inconsistency, |
| | | leading to significant quality |
| | | variation. |

It is noteworthy that the serum-free culture medium used in the present invention is particularly compatible with the production of SARS-COV-2 and MERS-COV virus-like particles (VLPs) using C6/36 (Aedes albopictus) cells, but may not be suitable for other mosquito-derived cell lines, such as AP-61 (Aedes pseudoscutellaris). This highlights the importance of optimizing culture conditions for specific cell lines. In other words, the serum-free culture medium employed in the present invention effectively supports cell growth and enables efficient viral protein expression, thereby facilitating the high-yield and high-quality production of VLP-based vaccines.

### 3. Culture Strategy

In practical implementation, the present invention adopts the following optimized culture strategy:
1. During routine cell passaging, serum-containing culture medium is used to maintain stable cell growth;
2. The culture is switched to serum-free medium only when VLP production is required;
3. During the 12-day VLP production period, the serum-free medium is replaced every 2 to 3 days.

This strategy offers the following advantages:
1. It ensures stable growth conditions for the cells under normal culture conditions;
2. During VLP production, it maintains basic cellular viability while ensuring that the production process occurs under serum-free conditions;
3. It eliminates the need for downstream serum removal steps, thereby reducing both culture and purification costs.

By adopting the aforementioned culture strategy, the present invention successfully balances the requirements of cell growth and VLP production, thereby providing an efficient, cost-effective, and reliable method for producing high-quality VLPs. This method not only significantly increases the yield and quality of VLPs, but also reduces production costs and potential contamination risks, thereby laying a solid foundation for large-scale manufacturing of coronavirus VLP vaccines.

### Example 2: Characterization of Coronavirus Virus-Like Particles (VLPs)

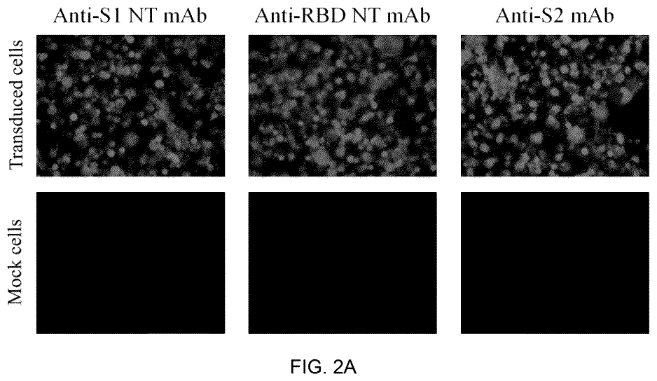

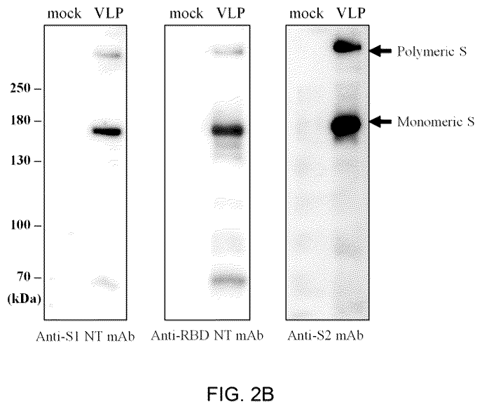

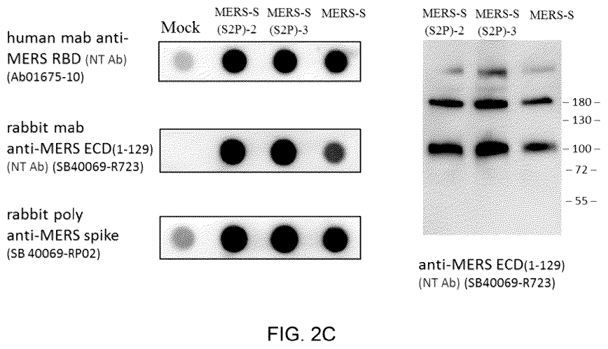

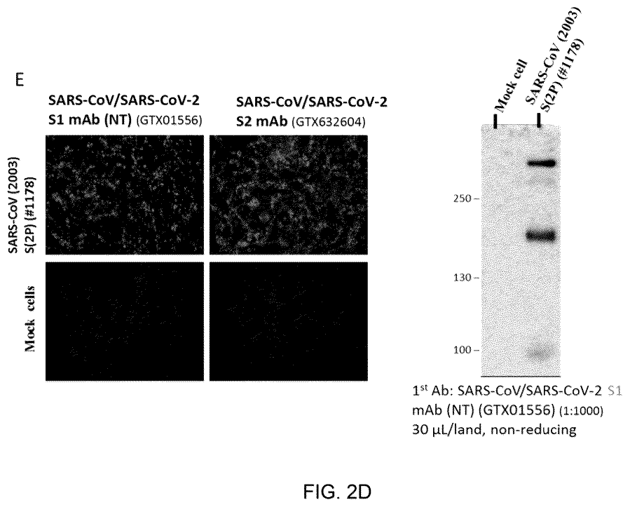

FIG. 2A shows the immunofluorescence staining results of SARS-COV-2 spike protein in transduced cells. The results indicate successful expression of the SARS-COV-2 spike protein in the transduced cells. Transduced cells (top row) and mock control cells (bottom row) were stained using Anti-S1 NT mAb (left column), Anti-RBD NT mAb (middle column), and Anti-S2 mAb (right column), with Hoechst 33342 used for nuclear staining.

FIG. 2B illustrates the secretion of the full-length monomeric spike protein. Western blot (WB) analysis was performed on the cultured supernatant samples of BacMos-monomeric S-transduced mosquito cells and control cells to detect S1 (left), RBD (middle) or S2 (right). The polymeric and monomeric spike proteins(S) are indicated by arrows on the right, and the molecular weights of the protein marker (kDa) are shown on the left.

FIG. 2C presents the results of the immunoblot assay, demonstrating the secretion of MERS-COV spike proteins (wild type and 2P mutant) in the transduced cells.

FIG. 2D shows the results of immunofluorescence staining and western blot analysis, indicating the expression of the SARS-COV (2003) spike protein (2P mutant) in the transduced cells.

FIGS. 2B and 2C demonstrate the expression and secretion of spike proteins from different coronaviruses (including SARS-COV-2, MERS-COV-2, and SARS-COV) in mosquito cells. These results were verified by immunofluorescence staining and western blot analysis, confirming that the corresponding coronavirus virus-like particles (VLPs) can be produced using the method described in Example 1.

### Example 3. Production and Purification of Coronavirus Virus-Like Particles (VLPs)

This example describes a method for producing and purifying coronavirus virus-like particles (VLPs) using the baculovirus/mosquito (BacMos) expression system.

### 1. Production of Coronavirus Virus-Like Particles (VLPs)

### 1.1 Expression System

In this example, coronavirus virus-like particles (VLPs) were produced using a baculovirus-transduced mosquito cell line (C6/36) expressing a single S protein (spike protein). Specifically, the BacMos system was used to express monomeric S protein, which corresponds to a prefusion-stabilized version of the SARS-COV-2 spike(S) protein containing double mutations at the furin cleavage site and the 2P position.

### 1.2 Assembly of Coronavirus Virus-Like Particles (VLPs)

By employing the multifunctional BacMos system to express a single spike protein, insect cells were able to effectively assemble and release spherical coronavirus virus-like particles (VLPs) approximately 40 nm in diameter, such as those corresponding to SARS-COV-2, MERS-COV, or SARS-COV (2003). Notably, unlike prior technologies, the present example demonstrates that using a single vector to express monomeric S protein via the BacMos system is sufficient to efficiently assemble and produce functional SARS-COV-2 VLPs capable of binding to the ACE2 (angiotensin-converting enzyme 2) receptor.

### 1.3 Production Advantages

The BacMos system used in this embodiment possesses a non-cytolytic nature, which facilitates extended harvest windows and simplifies downstream purification of coronavirus virus-like particles (VLPs). In addition, this approach may enable rapid adaptation to newly emerging variants of concern or other novel β-coronaviruses.

### 2. Purification of Coronavirus Virus-Like Particles (VLPs)

### 2.1 Sucrose Gradient Banding

In this embodiment, sucrose gradient ultracentrifugation was employed to separate and purify SARS-COV-2 virus-like particles (VLPs). The specific procedure was as follows:
(1) VLP samples were loaded onto a pre-formed sucrose density gradient;
(2) Ultracentrifugation was performed;
(3) After centrifugation, twelve gradient fractions were collected from top (Fraction 1) to bottom (Fraction 12).

### 2.2 Identification of Purified Coronavirus Virus-Like Particles (VLPs)

Western blot analysis was used to verify the presence of purified VLPs. The procedure included the following steps:
(1) All twelve fractions collected from the sucrose gradient ultracentrifugation were subjected to Western blot analysis;
(2) An anti-S1 monoclonal antibody (e.g., anti-S1 mAb) was used for detection;
(3) The presence of VLPs in each fraction was identified by observing the band signals and recording the corresponding fractions.

### 2.3 Transmission Electron Microscopy (TEM) Observation

To confirm the morphology and size of the purified coronavirus virus-like particles (VLPs), transmission electron microscopy (TEM) was employed. TEM enabled direct visualization of the spherical structure and particle size distribution of the VLPs, providing morphological validation of the purification results.

Through the production and purification procedures described in this embodiment, functional VLPs of SARS-COV-2, MERS-COV, and SARS-COV (2003) were successfully obtained. These coronavirus virus-like particles (VLPs) retain a stable prefusion conformation and present functional antigenic epitopes capable of binding to the ACE2 receptor, thereby establishing a solid foundation for subsequent vaccine development.

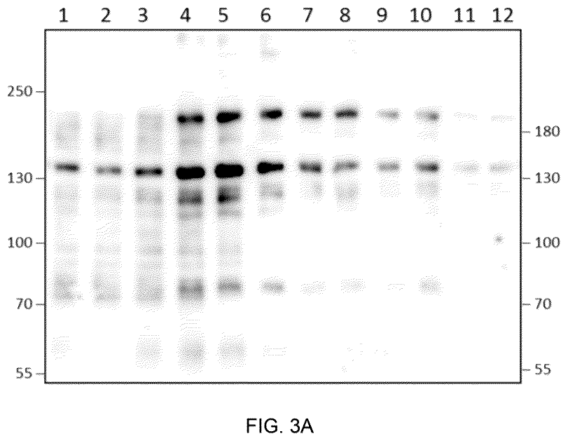

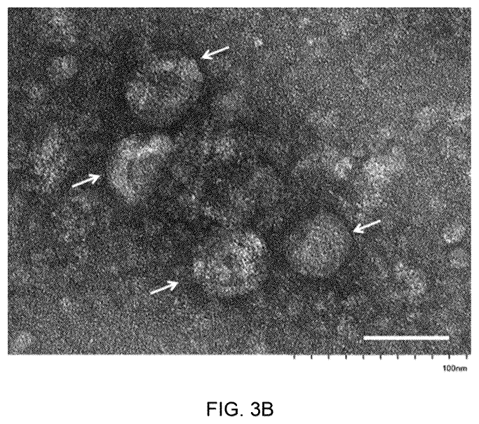

FIGS. 3A and 3B illustrate the characteristics of the coronavirus virus-like particles (VLPs) isolated in this embodiment. FIG. 3A shows the separation of SARS-COV-2 VLPs using a sucrose density gradient after ultracentrifugation. The collected fractions (from fraction 1 at the top to fraction 12 at the bottom) were analyzed by Western blot using an anti-S1 monoclonal antibody. The protein marker (M) sizes in kilodaltons (kDa) are indicated on both sides. FIG. 3B presents a transmission electron microscopy (TEM) image of partially purified SARS-COV-2 VLPs. The virus-like particles are indicated by white arrows. The scale bar represents 50 nm.

### Example 4: Functional Analysis of Coronavirus Virus-Like Particles (VLPs)

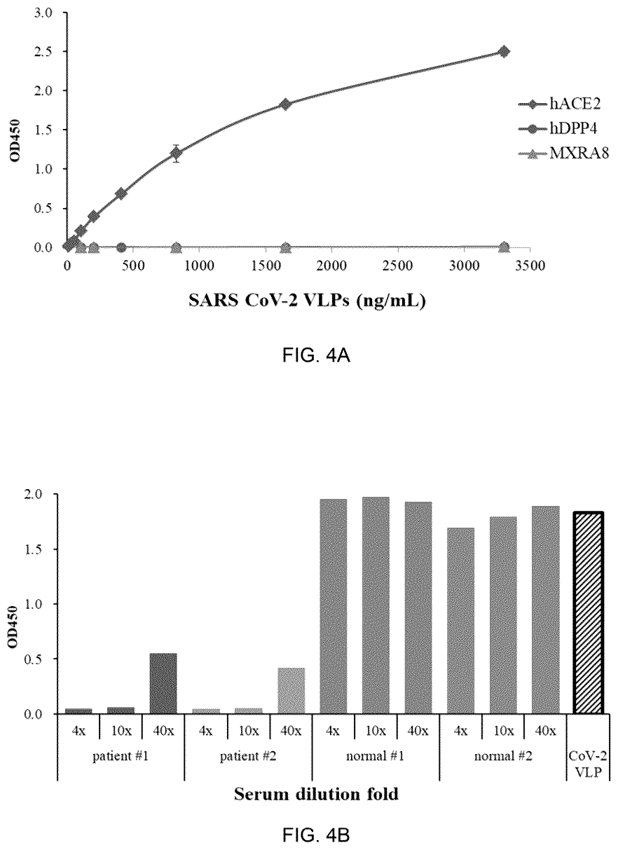

In this example, an ACE2 binding assay was conducted to demonstrate that the coronavirus virus-like particles (VLPs) produced and purified in Example 3 possess the ability to bind to the ACE2 receptor, indicating that functional antigenic epitopes are retained. FIG. 4A shows the binding capability of SARS-COV-2 VLPs to various receptors, including human ACE2 (hACE2), human DPP4 (hDPP4), and MXRA8. FIG. 4B presents the inhibition assay of SARS-COV-2 VLPs binding to human ACE2.

As shown in FIG. 4A, the results demonstrate the binding specificity of SARS-COV-2 virus-like particles (VLPs) to the ACE2 receptor. In this assay, wells were coated with recombinant human ACE2 (hACE2), human DPP4 (hDPP4), or MXRA8. Mouse sera and SARS-COV-2 VLPs were serially diluted and added to the pre-coated wells. The bound VLPs were detected using a neutralizing monoclonal antibody, NT mAb (GTX635791). The results in FIG. 4A show that the binding of SARS-COV-2 VLPs to hACE2 increased significantly with concentration, whereas binding to hDPP4 and MXRA8 was negligible. These results confirm the specific binding of the VLPs produced and purified in Example 3 to hACE2.

As shown in FIG. 4B, the figure presents the results of an inhibition assay evaluating the binding of SARS-COV-2 virus-like particles (VLPs) to the ACE2 receptor. Wells were coated with recombinant human ACE2 (hACE2) protein. The SARS-COV-2 VLPs were either left untreated or pre-incubated with serially diluted human sera from positive controls (sera from infected individuals 1 and 2) or negative controls (sera from healthy individuals 1 and 2). The resulting mixtures were then added to the hACE2-coated wells. The amount of VLPs bound to hACE2 was detected using the same method described in FIG. 4A. As shown in FIG. 4B, the untreated VLPs displayed strong binding to hACE2. In contrast, the VLPs pretreated with positive control sera exhibited a clear inhibitory effect on binding, whereas those pretreated with negative control sera showed no significant inhibition. These findings further confirm that the produced VLPs possess authentic viral antigenicity and can be specifically neutralized by antibodies present in the sera of infected individuals.

### Example 5-Immunogenicity of Coronavirus Virus-Like Particle (VLP) Vaccines

This example demonstrates the immunogenicity of coronavirus virus-like particle (VLP) vaccines administered via intranasal (IN) and intramuscular (IM) routes. By comparing different administration routes and dosages in an animal model, the immune responses induced by the VLP vaccines were systematically evaluated, thereby validating the feasibility and advantages of the VLPs as a mucosal immunization strategy.

### 1. Immunization Protocol

In this example, BALB/c mice were used as the animal model for immunization studies. The mice received intranasal (IN) or intramuscular (IM) administration of coronavirus virus-like particle (VLP) vaccines at doses of 2.5 μg or 10 μg per injection. The immunization schedule consisted of a prime dose followed by two booster doses, each administered at 21-day intervals, to evaluate the effects of dosage and route of administration on the elicited immune response.

### 2. Systemic Immune Responses

### 2.1 IgG Response

Following the second and third immunizations, serum levels of SARS-COV-2 S protein-specific total IgG, IgG1, and IgG2a antibodies were measured. The results demonstrated that the VLP vaccine effectively induced high-affinity IgG antibody production and elicited a balanced S-specific IgG2a (Th1) and IgG1 (Th2) immune response.

### 2.2 Neutralizing Antibody Response

A pseudovirus neutralization assay was conducted to evaluate the levels of neutralizing antibodies against the SARS-COV-2 wild-type (WT) strain in mouse sera. The results showed that both intranasal (IN) and intramuscular (IM) immunization routes effectively induced neutralizing antibody responses, with ID50 titers ranging from 100 to 1800. Notably, at a high immunization dose of 10 μg, the IN route induced a neutralizing antibody response 3.64 times higher than that of the IM route (mean ID50 titer for IN/IM=1800/500), demonstrating a significant advantage of intranasal administration in enhancing neutralizing immunity.

### 2.3 Cross-Neutralizing Activity

This study further evaluated the cross-neutralizing activity of sera against SARS-COV-2 variants, including Alpha, Delta, and Omicron strains. The results demonstrated that the VLPs vaccine induced broadly neutralizing antibodies capable of effectively targeting multiple SARS-COV-2 variants, indicating its potential for broad-spectrum protection.

### 3. Mucosal Immune Response

3.1 Secretory IgA (sIgA) Response

This study further evaluated the antigen-specific secretory IgA (sIgA) antibody response in bronchoalveolar lavage fluid (BALF) and intestinal samples. The results showed a significant increase in S-specific sIgA levels following VLPs vaccination. Notably, mucosal sIgA production in BALF was observed exclusively in mice immunized via the intranasal (IN) route, highlighting the essential role of IN administration in inducing mucosal immunity.

### 3.2 Mucosal Neutralizing Antibodies

Furthermore, mucosal neutralizing antibodies against SARS-COV-2 wild-type (WT) and Omicron pseudoviruses were detected in the bronchoalveolar lavage fluid (BALF). These findings indicate that the VLPs vaccine of the present invention can elicit neutralizing immune responses in the respiratory tract, potentially contributing to protective immunity against viral invasion.

### 4. Immunological Durability

This example evaluated the durability of the immune response induced by the vaccine. The results demonstrated that significant systemic antibody responses were still detectable in serum four months after the third immunization, indicating that the coronavirus virus-like particle (VLP) vaccine of the present invention possesses favorable immunological durability.

### In Vivo Protective Efficacy

This example evaluated the in vivo protective efficacy of the VLP vaccine using the K18-hACE2 transgenic mouse model. The results demonstrated that intranasal administration of the adjuvant-free SARS-COV-2 virus-like particle (VLP) vaccine effectively protected mice against a lethal challenge with SARS-COV-2 wild-type (WT) virus and also provided cross-protection against the Omicron variant, indicating the potential of the vaccine to prevent infections caused by different viral strains.

In summary, this example demonstrates that the coronavirus virus-like particle (VLP) vaccine—comprising the VLPs produced and purified as described in Example 3—exhibits excellent immunogenicity, capable of eliciting robust systemic and mucosal immune responses and providing effective protection against SARS-COV-2 and its variants. Notably, intranasal administration showed superior immunological outcomes compared to intramuscular injection, providing critical support for the development of next-generation COVID-19 vaccines.

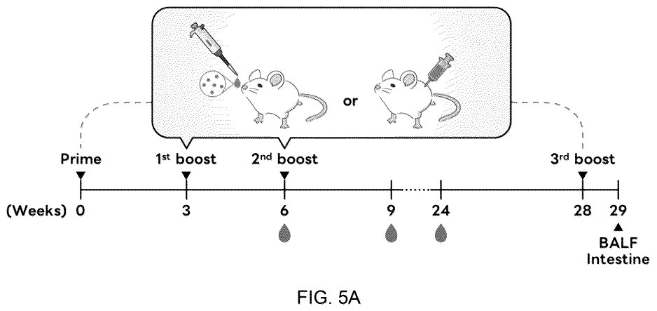

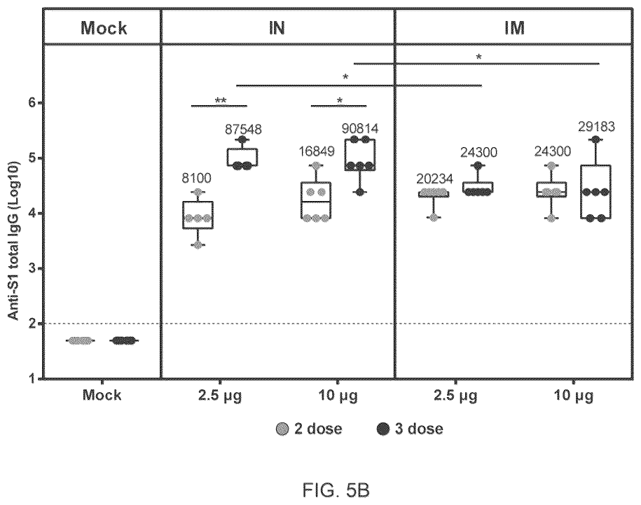

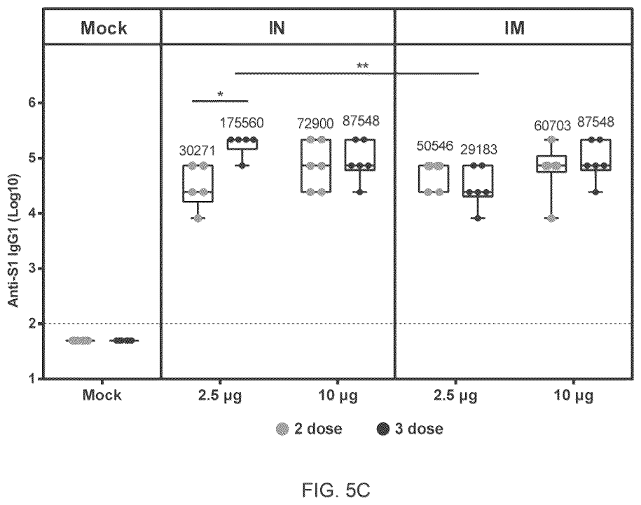

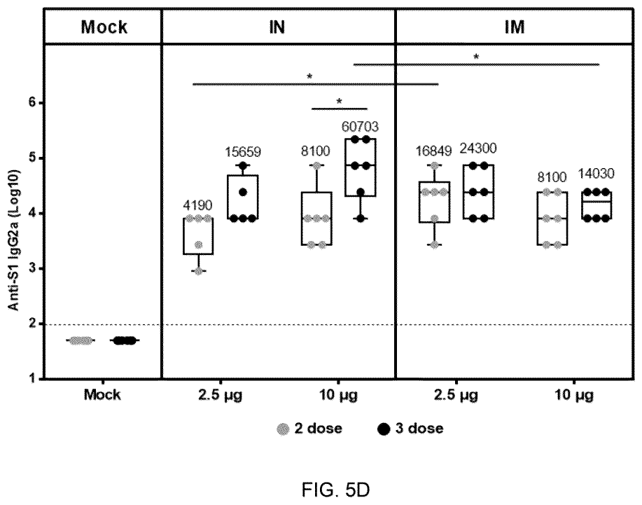

FIGS. 5A-5D illustrate the induction of S-specific antibodies—including total IgG, IgG1, and IgG2a—in mice following different routes of administration (intranasal [IN] and intramuscular [IM]) and varying numbers of doses. These results serve to evaluate the immune responses elicited by the adjuvant-free coronavirus virus-like particle (VLP) intranasal vaccine.

FIG. 5A illustrates the immunization schedule for BALB/c mice, including the routes of administration—intranasal (IN) and intramuscular (IM)—and the corresponding blood collection time points. FIGS. 5B-5D respectively show the SARS-COV-2 S-specific total IgG (FIG. 5B), IgG1 (FIG. 5C), and IgG2a (FIG. 5D) responses following the second (gray) and third (black) immunizations. The data indicate that both IN and IM routes effectively induced robust SARS-COV-2 S-specific antibody responses.

FIG. 5B illustrates the SARS-COV-2 S-specific total IgG antibody response. The vertical axis represents antibody titers, while the horizontal axis shows different immunization routes (IN or IM) and doses (2.5 μg or 10 μg). Gray bars correspond to the responses after the second immunization, and black bars represent those after the third immunization. The results demonstrate that both IN and IM routes elicited substantial IgG responses, with further enhancement observed following the third dose. Notably, the highest antibody levels were achieved after the third intranasal immunization at the high dose (10 μg).

FIG. 5C shows the response of SARS-COV-2 S-specific IgG1 antibodies. The vertical axis, horizontal axis, and color scheme (gray for the second dose and black for the third dose) are consistent with those in FIG. 5B. The results indicate that all immunized groups generated detectable levels of IgG1 antibodies. Both IN and IM routes successfully induced IgG1 responses; however, the IN group—particularly the high-dose group—exhibited stronger responses following the third immunization.

FIG. 5D illustrates the response of SARS-COV-2 S-specific IgG2a antibodies. The vertical axis, horizontal axis, and color representation are consistent with those in FIGS. 5B and 5C. The results show that all immunized groups elicited IgG2a antibody responses. Notably, the intranasal (IN) route, especially in the high-dose group, exhibited superior performance in inducing IgG2a responses, particularly after the third immunization.

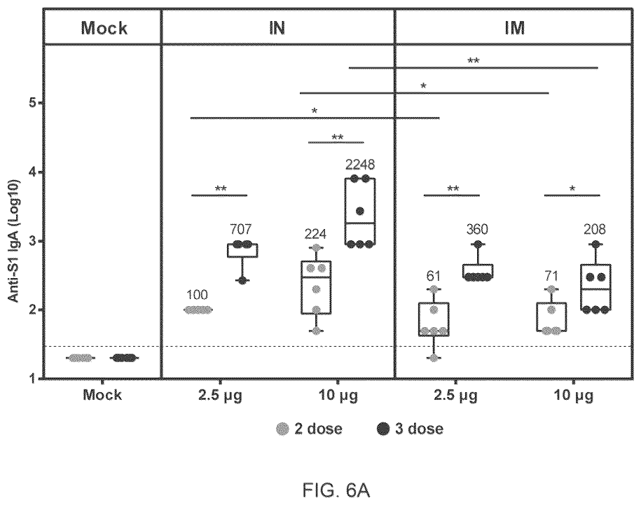

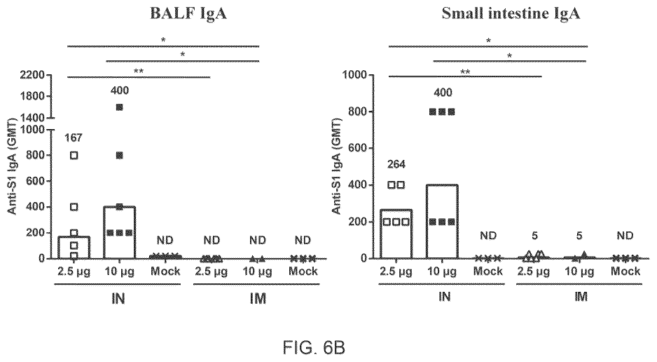

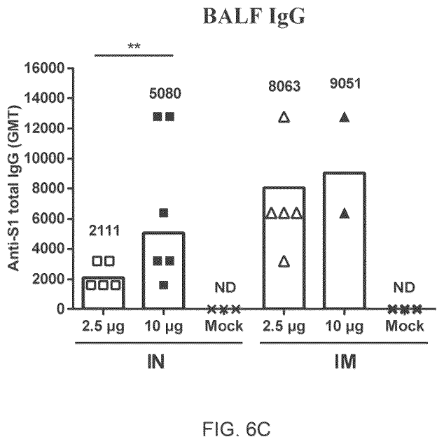

FIGS. 6A-6C demonstrate that intranasal (IN) immunization with SARS-COV-2 virus-like particles (VLPs) induces both mucosal and systemic immune responses. FIG. 6A shows the systemic SARS-COV-2 S-specific IgA responses detected in serum after the second (gray) and third (black) immunizations. FIG. 6B illustrates SARS-COV-2 S-specific secretory IgA (sIgA) responses observed in bronchoalveolar lavage fluid (BALF) (left panel) and the small intestine (right panel) following the second booster immunization. FIG. 6C presents the SARS-COV-2 S-specific total IgG response detected in BALF after the second booster. The results of FIGS. 6A-6C confirm that intranasal administration of SARS-COV-2 VLPs elicits robust mucosal and systemic immune responses. Notably, the IN route induced significant S-specific IgA responses in serum, BALF, and the small intestine. Importantly, only the IN route was capable of inducing mucosal sIgA production in BALF, which is critical for blocking viral infection at the respiratory tract entry point.

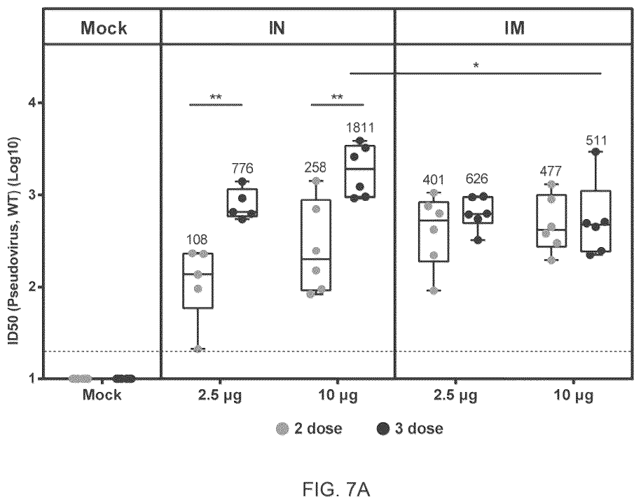

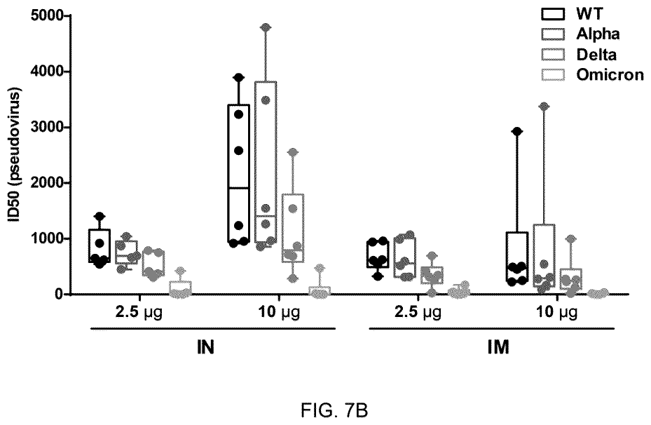

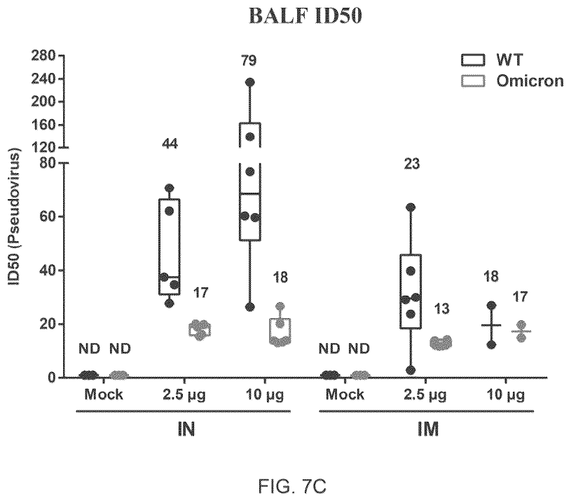

FIGS. 7A-7C illustrate the systemic and mucosal neutralizing antibody responses elicited by the SARS-COV-2 virus-like particle (VLP) vaccine. FIG. 7A shows the systemic neutralizing antibody responses against the wild-type (WT) virus strain following the second (gray) and third (black) immunizations. FIG. 7B presents the systemic neutralizing antibody responses against various variants of concern (VOCs) after the second booster immunization. FIG. 7C displays the mucosal neutralizing antibody responses in bronchoalveolar lavage fluid (BALF) targeting both the wild-type strain (black) and the Omicron variant (gray) after the third booster immunization. The results of FIGS. 7A-7C demonstrate that the SARS-COV-2 VLP vaccine can effectively induce robust systemic and mucosal neutralizing antibody responses. Notably, intranasal (IN) immunization exhibited superior performance in eliciting broadly neutralizing antibodies against the wild-type strain as well as multiple VOCs, including Alpha, Delta, and Omicron. Moreover, IN immunization also induced mucosal neutralizing antibodies in BALF against both the WT and Omicron strains, which is critical for preventing infection and transmission in the upper respiratory tract.

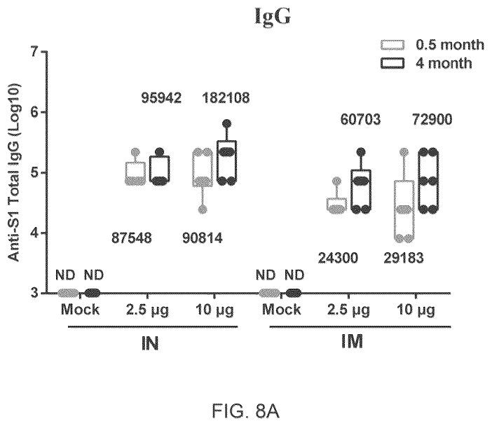

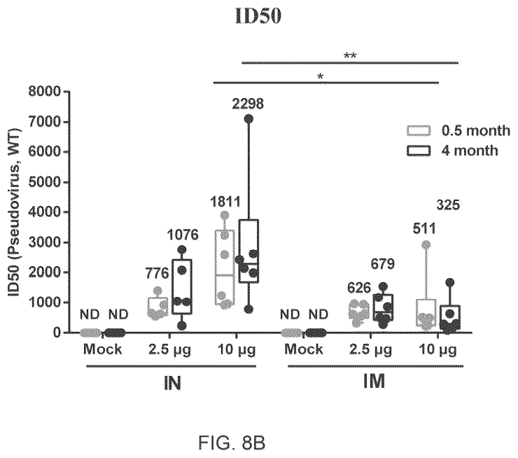

FIGS. 8A and 8B show the duration of systemic responses induced by intranasal (IN) or intramuscular (IM) immunization with SARS-COV-2 virus-like particles (VLPs). FIG. 8A illustrates the levels of systemic SARS-COV-2 S-specific total IgG at 0.5 and 4 months after the third immunization. FIG. 8B presents the corresponding neutralizing antibody responses at those same time points. The results of FIGS. 8A and 8B demonstrate that the SARS-COV-2 VLP vaccine is capable of eliciting durable systemic immune responses. Notably, significant levels of S-specific IgG and neutralizing antibodies were still detectable at 4 months post-final immunization. Furthermore, the IN route exhibited superior performance in maintaining long-term immune responses compared to the IM route, which is critical for ensuring sustained protective immunity.

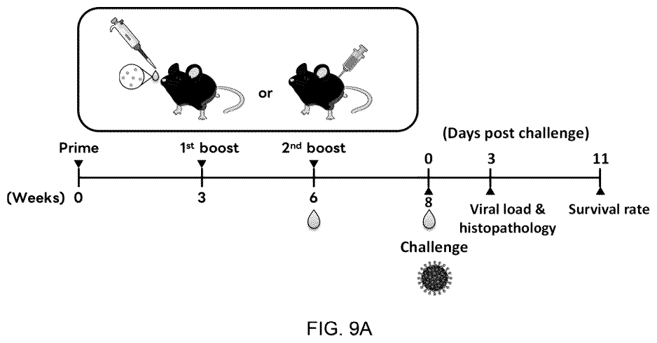

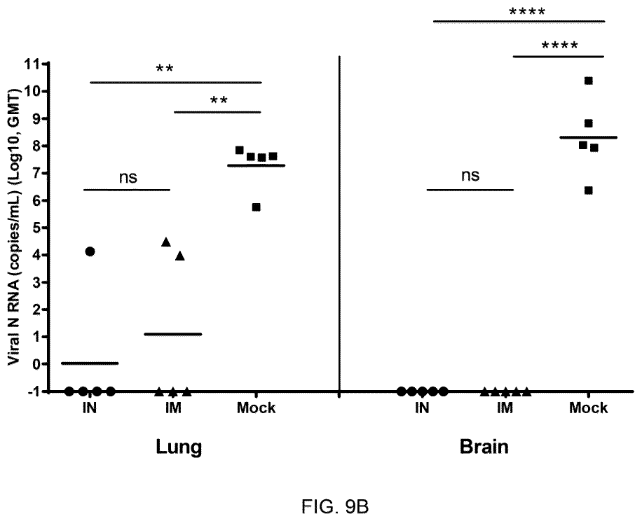

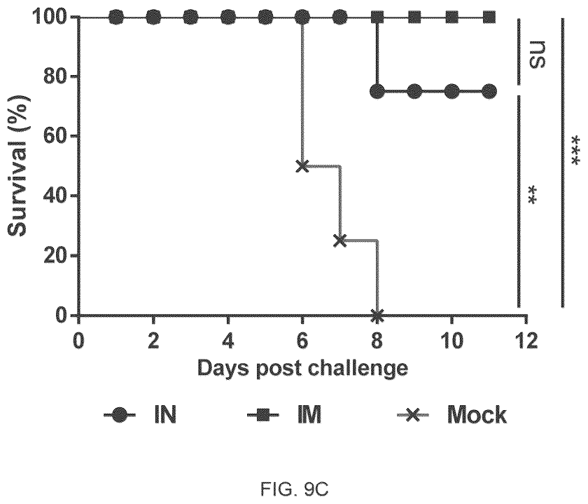

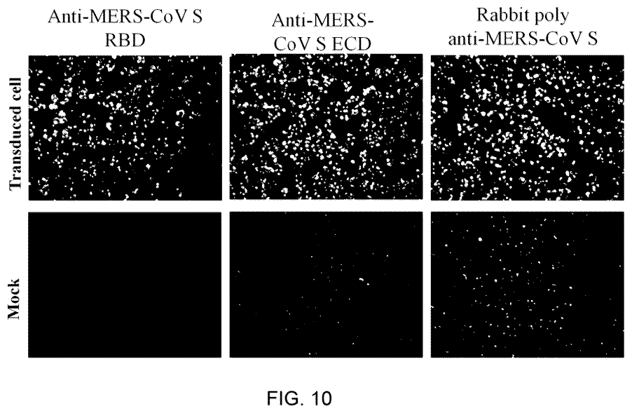

FIGS. 9A-9C demonstrate that intranasal vaccination with SARS-COV-2 virus-like particles (VLPs) effectively protects K18-hACE2 transgenic mice from SARS-COV-2 infection. FIG. 9A outlines the immunization and viral challenge schedule for the mice. Mice were immunized with 10 μg of SARS-COV-2 VLPs via either intranasal (IN) or intramuscular (IM) administration and received booster doses at 21 and 42 days post-immunization (dpi). Two weeks after the final immunization, K18-hACE2 mice were intranasally challenged with 20 μl PBS containing 104 PFU of SARS-COV-2 prototype virus. FIG. 9B shows the viral RNA loads in lung and brain tissues from mice in each group (IN VLPs, IM VLPs, and no vaccination (NV); n=5) on day 3 post-infection, as determined by qRT-PCR. FIG. 9C presents the survival curves of K18-hACE2 transgenic mice (n=4) in each group. The results of FIGS. 9A-9C clearly indicate that intranasal administration of SARS-COV-2 VLPs significantly reduces viral loads in both the lungs and brain, and improves survival, providing strong evidence that IN VLP vaccination confers robust protection and represents a promising strategy for COVID-19 prevention.

Through a series of experiments, the present invention demonstrated the efficacy and superiority of SARS-COV-2 virus-like particles (VLPs) produced using the BacMos system as an intranasal vaccine. The following sections provide a detailed description of the experimental results and their implications.

Conventional vaccines are primarily administered via intramuscular injection. However, many pathogens, including SARS-COV-2, invade the human body through mucosal surfaces, making intranasal vaccination an attractive alternative for mucosal vaccine delivery. Studies have also shown that intramuscular COVID-19 vaccines are insufficient to control SARS-COV-2 replication and shedding in the upper respiratory tract, potentially resulting in asymptomatic transmission or mild symptomatic infections. In contrast, intranasal vaccines exhibit the potential to induce sterilizing immunity against mucosal pathogens.

In this study, the inventors demonstrated that mosquito cells (C6/36) transduced with baculoviruses expressing a single S protein (spike protein) can effectively assemble and release spherical virus-like particles (VLPs) approximately 40 nm in diameter, including SARS-COV-2 (see FIGS. 2A, 2B, 3A, and 3B), MERS-COV (see FIG. 2C), and SARS-COV (2003) VLPs (see FIG. 2D). Moreover, the minimal molecular requirements for coronavirus VLP assembly may vary. For instance, in conventional systems, the co-expression of multiple structural proteins is necessary for VLP assembly in mammalian or other insect cell lines. In contrast, the present invention innovatively utilizes a BacMos system that expresses only monomeric S protein via a single vector, which is sufficient for the efficient assembly and production of functional SARS-COV-2 VLPs capable of binding the ACE2 receptor (see FIGS. 4A and 4B). This breakthrough not only simplifies the manufacturing process but also enables rapid adaptation to new variants of concern or other emerging β-coronaviruses. Additionally, the BacMos system used in the present invention exhibits a non-cytolytic nature, which facilitates extended harvest periods and simplifies downstream purification of VLPs.

In addition, unlike intramuscular injection, the intranasal vaccine provided by the present invention is needle-free and does not require a sterile environment for administration. This feature improves patient compliance, reduces the need for specialized training, minimizes the risk of blood-borne diseases, and, more importantly, induces both mucosal and systemic immune responses. Although nasal vaccination has historically faced challenges such as mucus barriers, epithelial barriers, and the lack of compatible adjuvants, the nanoparticle platform offered by the present invention provides an innovative solution. Specifically, the nanoparticle-based vaccine enhances antigen permeability across the nasal mucosal barrier, ensuring delivery to immune-inductive sites within the nasal cavity. It also resists degradation, prolongs antigen retention within the nasal environment, and stimulates long-lasting immune responses. Furthermore, the nanoparticles possess inherent adjuvant properties that enhance the immunogenicity of subunit vaccines.

In the present invention, the inventors developed novel insect-derived SARS-COV-2 virus-like particles (VLPs). These VLPs were generated from a construct based on monomeric spike protein-specifically, a pre-fusion version of the SARS-COV-2 spike(S) protein containing dual mutations at the furin cleavage site and the 2P positions. The VLPs were evaluated as promising intranasal (IN) vaccine candidates. Experimental results demonstrated that the VLP vaccine could induce both mucosal and systemic immune responses in a murine model. This dual immunogenicity indicates the potential significance of these VLPs in preventing SARS-COV-2 infection and transmission.

The self-adjuvanted virus-like particles (VLPs) of the present invention, when administered intranasally (IN) or intramuscularly (IM) in two doses (2.5 μg or 10 μg per dose), effectively elicited high-affinity IgG titers (see FIG. 5B), balanced S-specific IgG2a/IgG1 responses, and potent neutralizing antibody responses against wild-type (WT) SARS-COV-2 (ID50 titers ranging from 100 to 1800; see FIG. 7A). Notably, after three doses, the IN group exhibited significantly enhanced titers of total S-specific IgG, IgG2a, IgG1, and neutralizing antibodies (see FIGS. 5C, 5D, and 7A), whereas the IM group did not show significant enhancement after the third dose. These results underscore the superiority of the intranasal route of VLP vaccine administration in the present invention. In addition, the Th1-biased response induced by the vaccine—as indicated by elevated IgG2a levels (see FIG. 5D)—may be associated with enhanced CD8⁺ T cell-mediated immune surveillance, thereby promoting viral clearance. Importantly, the neutralizing antibody (NT) responses induced by the high-dose IN immunization were 3.6-fold greater than those induced by IM immunization (mean ID50 titer for IN/IM=1800/500; see FIG. 7A), further demonstrating the superiority of the intranasal VLP vaccine in inducing robust immune responses.

Secretory IgA (sIgA) antibodies are the principal effectors of mucosal immunity and have demonstrated superior efficacy over IgG in combating SARS-COV-2 infection. Studies have shown that resistance to mucosal infection by SARS-COV-2 in animal models is closely associated with the presence of sIgA. In the present invention, the inventors found that intranasal (IN) immunization with virus-like particles (VLPs) not only elicited systemic neutralizing antibodies in the serum against wild-type (WT) S and variant (VOCs: Alpha, Delta, and Omicron) S pseudoviruses (see FIG. 7B), but also induced mucosal neutralizing antibody responses against WT-S and Omicron-S pseudoviruses in bronchoalveolar lavage fluid (BALF) (see FIG. 7C). These findings underscore the potential of the VLP vaccine of the present invention to induce broad and effective immune protection.

The adjuvant-free intranasal prototype SARS-COV-2 virus-like particle (VLP) vaccine of the present invention effectively protected K18-hACE2 transgenic mice from lethal SARS-COV-2 challenge (see FIGS. 9A, 9B, and 9C), and also conferred cross-protection against infection by the SARS-COV-2 Omicron variant (data not shown). These findings demonstrate that intranasal (IN) immunization with VLPs can establish both systemic and mucosal immunity, thereby providing critical protection against SARS-COV-2 variants of concern (VOCs). Notably, the ability of the IN vaccine to induce broadly neutralizing antibodies makes it especially attractive for halting the spread of new COVID-19 variants. Specifically, IN immunization with VLPs can elicit high levels of neutralizing IgA in the upper respiratory tract, offering a first line of defense against infection and transmission of Omicron subvariants.

It is noteworthy that although both intranasal (IN) and intramuscular (IM) routes of administration of SARS-COV-2 virus-like particles (VLPs) can elicit immune responses, the IN route induces a markedly stronger immunogenic effect. In particular, mucosal secretory IgA (sIgA) in bronchoalveolar lavage fluid (BALF) was only stimulated via the IN route (see FIGS. 6A, 6B, and 6C). Studies have shown that sIgA plays a critical role by interfering with viral infection at both the entry and exit sites. In contrast, IM administration of wild-type SARS-COV-2 VLPs failed to generate detectable neutralizing titers against the SARS-COV-2 wild-type strain (data not shown).

However, when SARS-COV-2 virus-like particles (VLPs) with a stabilized prefusion conformation are administered via intranasal (IN) or intramuscular (IM) routes, potent neutralizing antibodies against SARS-COV-2 wild-type (WT) and variants of concern (VOCs), including Alpha and Omicron strains, can be observed. These findings further demonstrate that the SARS-COV-2 VLPs S (2P) candidate vaccine of the present invention has the potential to prevent infections caused by various viral variants.

The present invention further utilizes the modulation of physicochemical properties of nanoparticles to enhance mucosal immune responses. This includes controlling the particle size within the range of 20 to 200 nanometers, employing positively charged nanoparticles, and directing cell targeting through carbohydrate-based structures. The virus-like particles (VLPs) of the present invention exhibit these characteristics, which facilitate effective antigen transport across the nasal mucosal barrier and incorporate mucosal adjuvant activity to enhance immune induction. Specifically, the mosquito cell-derived VLPs used in this invention are associated with specific ligands that enable targeting of microfold cells (M cells) within nasal and bronchus-associated lymphoid tissues, thereby promoting epithelial translocation to immune activation sites. Furthermore, the highly repetitive surface antigenic epitopes presented by these VLPs correspond to pathogen-associated molecular patterns and engage with pattern recognition receptors on innate and adaptive immune cells (such as Toll-like receptors, NOD-like receptors, and RIG-I-like receptors), resulting in potent immunogenicity in vivo.

The SARS-COV-2 virus-like particles (VLPs) provided in the present invention possess inherent immunogenicity and can function as multivalent scaffolds for three-dimensional antigen presentation, while also serving as adjuvants to enhance immune responses. By incorporating specific ligands, the functional VLPs of the present invention can effectively adhere to the nasal epithelium, traverse the epithelial barrier, and induce both robust mucosal immunity and sustained systemic immune responses (see FIG. 8A and FIG. 8B), thereby demonstrating their potential as intranasal vaccine carriers.

In summary, the present invention provides a novel method for producing functional SARS-COV-2 virus-like particles (VLPs), which exhibit inherent self-adjuvanting properties and excellent safety. These VLPs are generated using a mosquito cell system transduced with a monomeric S protein derived from the wild-type strain via the innovative BacMos platform developed in this invention. Experimental results demonstrate that intranasal administration of the VLP vaccine induces both S-specific IgG and IgA antibodies, as well as broadly neutralizing antibodies effective against various SARS-COV-2 variants. More importantly, this immunization strategy elicits mucosal cross-neutralizing activity against the Omicron variant and establishes long-lasting systemic immunity. Notably, the intranasal route of administration in the present invention proves superior to conventional intramuscular injection in terms of eliciting immune responses.

In view of the critical role of mucosal transmission in SARS-COV-2 infection and spread, the present invention successfully translates intranasal administration of self-adjuvanting SARS-COV-2 virus-like particles (VLPs) into a clinically promising mucosal vaccination strategy, with comprehensive scientific data to support its feasibility. The immunization approach disclosed herein not only enhances the efficacy of SARS-COV-2 prevention and control, but also offers a novel framework for protecting against future emerging coronavirus infections.

### Example 6—Production and Characterization of MERS-COV Virus-Like Particles (VLPs)

This example describes the use of the baculovirus/mosquito cell (BacMos) system for the production of MERS-COV virus-like particles (VLPs), and presents the corresponding characterization results of the generated VLPs.

### 1. Production of MERS-COV Virus-Like Particles (VLPs)

In this example, a recombinant baculovirus expressing the MERS-COV spike(S) protein was constructed using the BacMos system. The spike protein was designed as a prefusion-stabilized version incorporating mutations at the furin cleavage site and two proline substitutions (2P). The recombinant virus also contained the mosquito-specific hr1 pag1 promoter, the JEV prM signal peptide, the RhPV 5′ untranslated region (5′-UTR) internal ribosome entry site (IRES), an eGFP reporter gene, and a translational stop codon to facilitate expression regulation and marker tracking.

### 2. Expression and Secretion of MERS-COV Virus-Like Particles (VLPs)

As shown in FIG. 10, immunofluorescence staining confirmed that the MERS-COV spike protein was successfully expressed in the transduced C6/36 mosquito cells. Specifically, both the transduced cells (top row) and control cells (bottom row) were stained with three types of antibodies: the left column used an anti-MERS-COV S RBD monoclonal antibody (targeting the receptor-binding domain (RBD) of the spike protein; neutralizing antibody), the middle column used an anti-MERS-COV S ECD monoclonal antibody (targeting the extracellular domain (ECD) of the spike protein; neutralizing antibody), and the right column used a rabbit polyclonal anti-MERS-COV S antibody. Cell nuclei were counterstained with Hoechst 33342. The scale bar represents 30 μm.

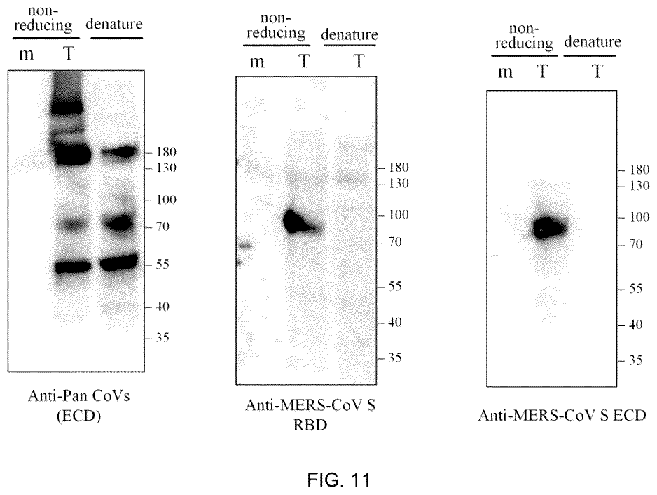

The secretion of the MERS-COV spike protein is illustrated in FIG. 11. Western blot analysis was performed on culture supernatants collected from BacMos-monomeric MERS-COV S-transduced mosquito cells (T) and mock-transduced control cells (m), under non-reducing and denaturing conditions. Three antibodies were used for detection: an anti-Pan CoVs antibody (targeting the extracellular domain (ECD) of coronavirus spike proteins), an anti-MERS-COV S RBD monoclonal antibody (targeting the receptor-binding domain), and an anti-MERS-COV S ECD monoclonal antibody (targeting the extracellular domain). The detected protein bands demonstrate the secretion of MERS-COV spike protein under different analytical conditions.

As shown in FIG. 10, successful expression of MERS-COV spike protein in C6/36 cells was clearly demonstrated by distinct fluorescence signals detected using three different antibodies targeting the RBD region, ECD region, and full-length S protein. FIG. 11 further shows that spike protein signals were detected exclusively in the culture supernatant of transduced cells (T), but not in the mock-transduced control cells (m), confirming that the transduced C6/36 cells successfully secreted monomeric full-length MERS-COV spike protein. Moreover, this spike protein was detectable by antibodies against different structural regions (RBD and ECD). Importantly, the presence of a signal using an anti-Pan CoVs antibody indicates that the secreted spike protein exhibits cross-reactive coronavirus features, demonstrating that the spike protein generated by this system retains its biological functionality and confirms the efficacy of this platform for constructing functional MERS-COV spike proteins.

### 3. Purification and Characterization of MERS-COV VLPs

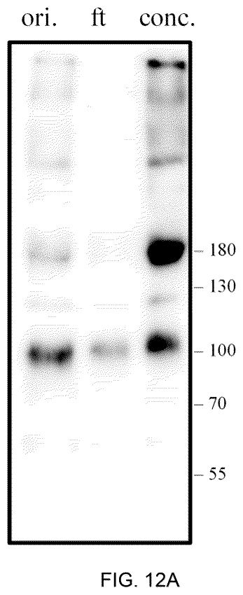

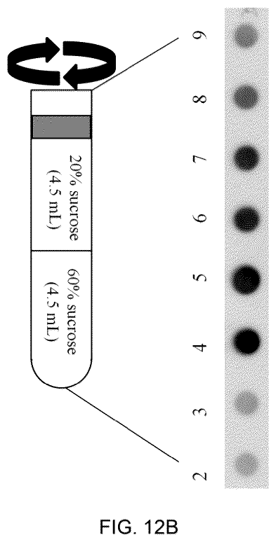

As shown in FIG. 12A, MERS-COV VLPs were concentrated using a tangential flow filtration (TFF) system with the MidiKros Module equipped with a 300,000 molecular weight cut-off membrane (Spectrum Repligen, Rancho Dominguez, CA, USA). Samples collected at each TFF stage—namely the original input (ori), flow-through (ft), and post-concentration fraction (conc.)—were subjected to Western blot analysis. The detection was performed under non-reducing conditions using a neutralizing monoclonal antibody targeting the extracellular domain (ECD) of the MERS-COV spike protein (NT Ab), to confirm the presence of the target spike protein in the purified MERS-COV VLP preparations.

As shown in FIG. 12B, the concentrated VLPs were further loaded onto a sucrose gradient (20% to 60%) for separation. After ultracentrifugation at 250,000×g for 3 hours, eight fractions were collected sequentially from the top to the bottom of the gradient. Each fraction was subjected to dot blot analysis using a neutralizing monoclonal antibody targeting the extracellular domain (ECD) of the MERS spike protein (Anti-MERS ECD, NT Ab), to determine the distribution of MERS-COV VLPs within the gradient.

Based on the results shown in FIGS. 12A and 12B, the present invention successfully established an efficient method for the concentration and purification of MERS-COV virus-like particles (VLPs). Initially, the use of a tangential flow filtration (TFF) system effectively concentrated the VLPs, as evidenced by the Western blot analysis in FIG. 12A, where the concentrated sample (conc.) exhibited the strongest spike protein signal. Subsequently, sucrose gradient ultracentrifugation further separated the VLPs, with dot blot analysis in FIG. 12B indicating that the particles were primarily distributed in fractions 4 through 7. This two-step process not only achieved substantial concentration but also enabled high-purity separation, yielding high-quality MERS-COV VLPs suitable for vaccine development and related studies. In other words, the method demonstrates strong potential for application in the production and purification of coronavirus VLPs.

### Example 7: Production and Characterization of SARS-COV VLPs

This example describes the use of the baculovirus/mosquito cell (BacMos) system for the production of SARS-COV virus-like particles (VLPs), along with their expression and secretion profiling. The results demonstrate that this method enables the efficient generation of biologically active SARS-COV VLPs, providing a solid experimental foundation for subsequent vaccine development and related research.

### 1. Production of SARS-COV VLPs

In this example, a recombinant baculovirus encoding the SARS-COV spike(S) protein gene was constructed using the BacMos system. The spike gene was designed in a prefusion-stabilized form, incorporating a furin cleavage site mutation and a 2P mutation to enhance structural stability and immunogenicity. The recombinant construct also included regulatory and auxiliary elements such as the hr1 pag1 mosquito promoter, JEV prM signal peptide, RhPV 5′-UTR IRES, an eGFP reporter gene, and a translation stop codon to ensure efficient expression in C6/36 mosquito cells.

### 2. Expression and Detection of SARS-COV VLPs

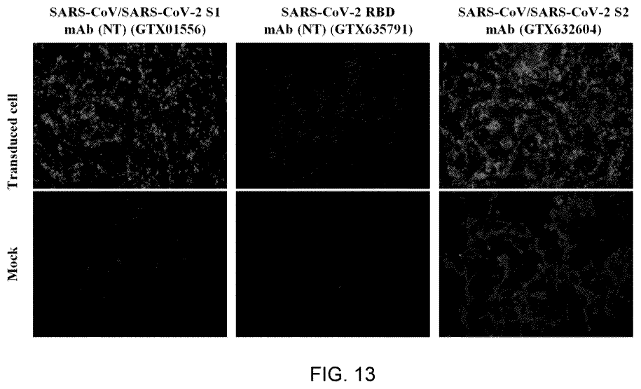

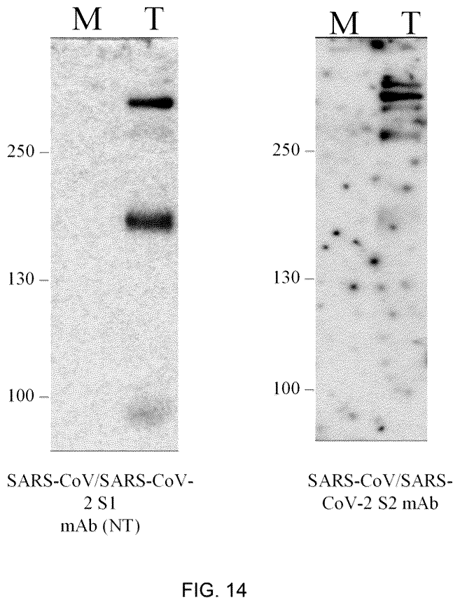

As shown in FIG. 13, immunofluorescence staining confirmed the successful expression of SARS-COV spike(S) protein in transduced AP-61 mosquito cells. The upper row of images represents transduced cells, while the lower row shows non-transduced control cells. Cells were stained with three different monoclonal antibodies: Anti-SARS-COV/SARS-COV-2 S1 (left column, neutralizing antibody), Anti-SARS-COV-2 RBD (middle column), and Anti-SARS-COV/SARS-COV-2 S2 (right column). Cell nuclei were counterstained with Hoechst 33342 for localization. The scale bar represents 30 μm. These results demonstrate that the SARS-COV spike protein was successfully expressed in insect cells and can be recognized by antibodies targeting various structural domains of the protein.

### 3. Secretion of SARS-COV VLPs

As shown in FIG. 14, the secretion of SARS-COV spike(S) protein from transduced mosquito cells into the culture supernatant was successfully confirmed. Western blot analysis was conducted using samples collected from BacMos-transduced mosquito cells (T) expressing monomeric SARS-COV S protein and from non-transduced control cells (M). Under non-reducing conditions, protein detection was performed using two monoclonal antibodies: Anti-SARS-COV/SARS-COV-2 S1 (left panel, neutralizing antibody) and Anti-SARS-COV/SARS-COV-2 S2 (right panel). Molecular weight markers (kDa) are shown on the left side of the blot. The results clearly demonstrate the presence of secreted spike protein in the supernatant of transduced cells, indicating that the BacMos system enables efficient secretion of SARS-COV structural proteins.

Taken together, these findings confirm that the method provided in the present invention successfully enabled the expression of SARS-COV spike protein in AP-61 cells, and that the expressed protein was efficiently secreted into the culture medium. Both immunofluorescence staining and Western blot analysis clearly demonstrated the expression and secretion of the spike protein, further validating that the invention can effectively produce biologically active SARS-COV virus-like particles (VLPs).

### Example 8: Optimization of SARS-COV-2 VLP Production

This example aims to evaluate the production efficiency of SARS-COV-2 virus-like particles (VLPs) under different insect cell lines and culture conditions, with the goal of identifying optimal production parameters. A systematic experimental approach was employed to assess the effects of various factors on VLP yield and quality, thereby supporting downstream applications in vaccine manufacturing.

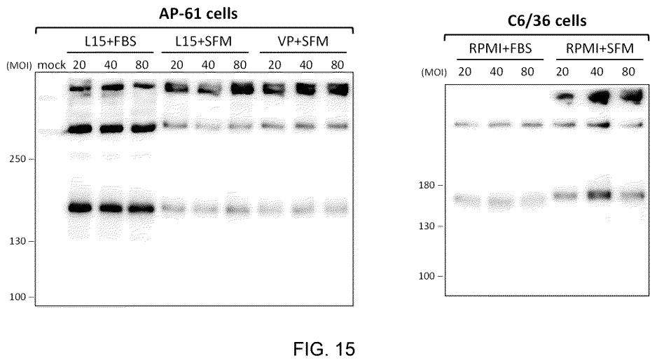

As shown in FIG. 15, the present invention conducted a comparative analysis of SARS-COV-2 (S2P) virus-like particle (VLP) production efficiency in different insect cell lines. Specifically, for AP-61 cells, three distinct culture media conditions were evaluated: L15 supplemented with fetal bovine serum (L15+FBS), L15 supplemented with serum-free medium (L15+SFM), and VP medium supplemented with serum-free medium (VP+SFM). In each condition, VLP expression levels were assessed under three multiplicities of infection (MOI): 20, 40, and 80. For C6/36 cells, two media formulations were tested—RPMI supplemented with FBS (RPMI+FBS) and RPMI with SFM (RPMI+SFM)—again under MOIs of 20, 40, and 80. These experiments provided critical data for selecting the optimal cell line and culture conditions to facilitate large-scale production of VLPs.

Western blot analysis was further performed to quantify the production of SARS-COV-2 VLPs in the culture supernatants. The analysis was conducted under non-reducing conditions, using a monoclonal neutralizing antibody (NT Ab) specific to the receptor-binding domain (RBD) of the SARS-COV-2 spike protein. Signal detection was carried out using the high-sensitivity Amersham ECL Select reagent (RPN2235), allowing effective visualization even at low protein concentrations.

The results demonstrated that the production yield of SARS-COV-2 (S2P) VLPs varied significantly depending on the cell line, culture medium, and multiplicity of infection (MOI). Among all tested conditions, the C6/36 cell line cultured in RPMI medium supplemented with serum-free medium (SFM) exhibited the strongest protein expression signals, indicating the highest VLPs yield. Furthermore, under RPMI+SFM conditions, increasing the MOI from 20 to 40 to 80 resulted in a progressive enhancement of signal intensity in Western blot analysis, suggesting a positive correlation between MOI and VLPs production. Collectively, the optimal condition for large-scale production of SARS-COV-2 (S2P) VLPs was identified as: C6/36 cells, RPMI+SFM medium, and MOI of 80. This optimized combination provides an important basis for high-yield production of VLP-based vaccine candidates.

### Example 9—Optimization of MERS-COV VLPs Production

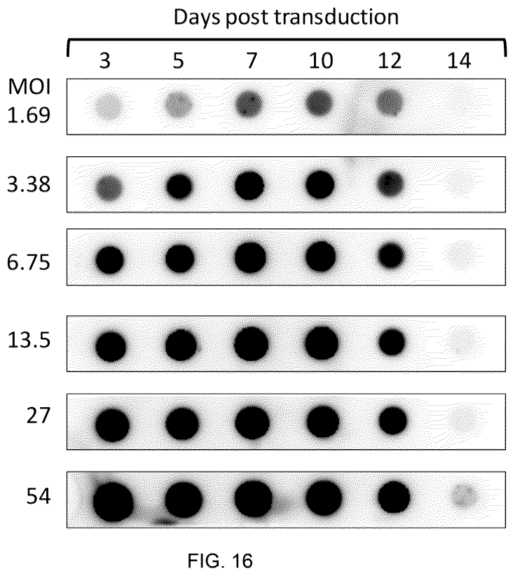

This example demonstrates the optimization of MERS-COV VLPs production. As shown in FIG. 16, the production of MERS-COV (S2P) VLPs was optimized using C6/36 cells cultured in serum-free medium (SFM). Various multiplicities of infection (MOIs: 1.69, 3.38, 6.75, 13.5, 27, and 54) and different harvest time points (3, 5, 7, 10, 12, and 14 days post-transduction) were evaluated. The production yield of VLPs was analyzed using an Anti-Pan CoVs ECD antibody, and the signals were visualized with the ECL Select detection system with a 3-second exposure time.

The results showed that when using serum-free medium (SFM) in C6/36 cells, the production yield of MERS-COV VLPs varied with increasing MOI and extended harvest time. These findings provide important references for large-scale production of MERS-COV VLPs. Specifically, under the condition of MOI 54, VLP production was clearly observed as early as day 3 post-transduction and remained consistently detectable through day 14, demonstrating a long-term stable production profile. This characteristic not only helps to increase the overall yield but may also reduce production costs, making it highly valuable for industrial-scale manufacturing.

### Example 10: Optimization of SARS-COV VLPs Production

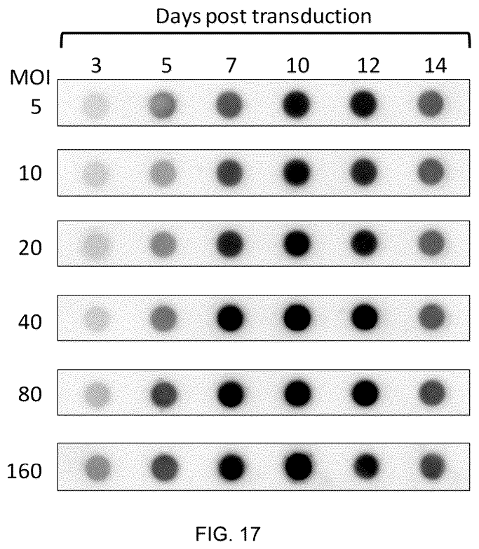

This example demonstrates the optimization results for the production of SARS-COV VLPs. As shown in FIG. 17, the present invention optimized the production of SARS-COV (S2P) VLPs using serum-free medium (SFM) in C6/36 cells. Different MOIs (5, 10, 20, 40, 80, and 160) and different harvest times (days 3, 5, 7, 10, 12, and 14 post-transduction) were tested. Detection was performed using Anti-SARS-COV/SARS-COV-2 S antibody, and the ECL select exposure time was set to 1 minute.

The results showed that when using serum-free medium (SFM) in C6/36 cells, different MOIs and harvest times had a significant impact on the yield of SARS-COV VLPs. These data provide an important reference for large-scale production of SARS-COV VLPs. Specifically, the cumulative yield of VLPs increased significantly with extended culture duration, particularly under MOI 80 and 160 conditions, and this trend persisted through day 14, exhibiting a long-term and stable production profile. This continuous production pattern not only facilitates optimization of harvest strategies and improvement of total yield but also contributes to enhancing production efficiency and reducing costs.

### Example 11: Protective Immunization Study of SARS-COV-2 VLPs in a Hamster Model

This example evaluated the immunogenicity and protective efficacy of SARS-COV-2 VLPs in a hamster model.

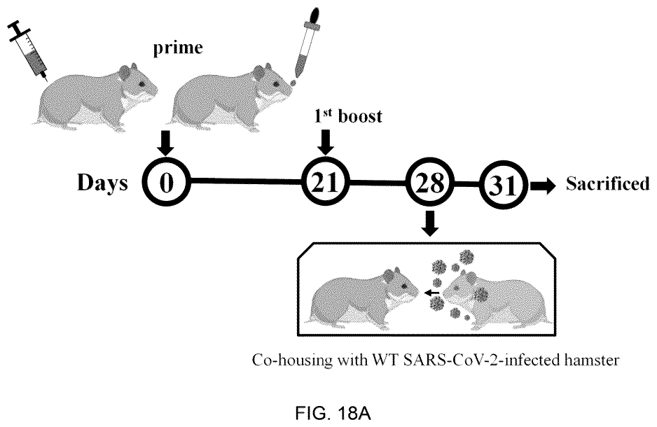

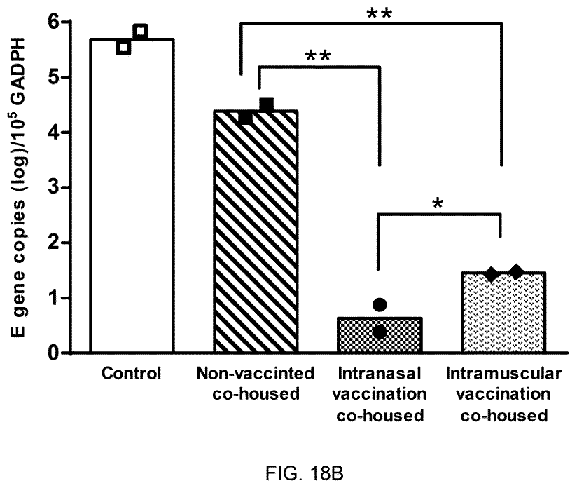

As shown in FIG. 18A, the experimental design was as follows: hamsters (N=2) were administered 10 μg of VLPs either intranasally (IN) or intramuscularly (IM), or given intranasal PBS as a negative control. One week after completing two doses of vaccination, the vaccinated hamsters were co-housed with hamsters that had been challenged with wild-type (WT) SARS-COV-2 for one day to conduct the challenge study.

FIG. 18B shows the viral RNA levels in the lungs of hamsters on day 3 post-infection (dpi). The results are categorized as follows:
1. Non-vaccinated co-housed group: hamsters that did not receive vaccination and were co-housed with challenged hamsters;
2. Intranasal vaccination co-housed group: hamsters vaccinated with SARS-COV-2 VLPs via the intranasal (IN) route and co-housed with challenged hamsters;
3. Intramuscular vaccination co-housed group: hamsters vaccinated with SARS-COV-2 VLPs via the intramuscular (IM) route and co-housed with challenged hamsters;
   4. Control group: challenged hamsters used as the infection source, measured on day 4 post-infection.

The results demonstrated that, compared to the control group, hamsters vaccinated with SARS-COV-2 VLPs—whether via the intranasal (IN) or intramuscular (IM) route—showed a significant reduction in viral RNA levels in the lungs. These findings indicate that SARS-COV-2 VLPs administered either intranasally or intramuscularly can induce protective immunity against SARS-COV-2 in the hamster model. Notably, the intranasal vaccination group exhibited lower viral RNA levels in the lungs compared to the intramuscular vaccination group, suggesting that the IN route provides superior protective efficacy.

Specifically, FIG. 18B illustrates the viral RNA levels in the lungs of hamsters from the control group, the non-vaccinated co-housing group, the intranasal vaccination co-housing group, and the intramuscular vaccination co-housing group. By comparing the viral RNA levels across the different groups, it is evident that VLP vaccination-particularly via the intranasal route-significantly reduced viral replication.

These results further confirm the effectiveness of the SARS-COV-2 VLP vaccine of the present invention, particularly demonstrating the clear superiority of the intranasal administration route in reducing viral loads in the upper respiratory tract. This feature not only helps to alleviate infection symptoms in individuals but may also reduce the risk of viral transmission, providing a new strategy for effectively controlling the spread of SARS-COV-2.

In summary, this embodiment confirmed the protective efficacy of the SARS-COV-2 VLP vaccine of the present invention in a hamster model, particularly highlighting the advantages of the intranasal administration route. The experimental results clearly demonstrated that, compared with the unvaccinated control group, hamsters vaccinated with the VLP vaccine—especially those in the intranasal administration group—exhibited significant protection against SARS-COV-2 infection. These findings provide important experimental evidence supporting the clinical potential of the present invention and offer new directions for the development of more effective COVID-19 prevention strategies.

The foregoing description has fully and clearly set forth a method for preparing coronavirus virus-like particles (VLPs) and a vaccine comprising such VLPs according to the present invention. It must be emphasized that the above detailed description merely provides specific explanations of feasible embodiments of the present invention and is not intended to limit the scope of the present invention. Any equivalent implementations or modifications made without departing from the technical spirit of the present invention shall fall within the scope of the present invention.

What is claimed is:

1. A method for preparing a coronavirus virus-like particle (VLP), comprising the steps of:
a) providing a recombinant baculovirus carrying a coronavirus spike(S) protein gene;
b) transducing an insect cell with the recombinant baculovirus from step (a); and
c) culturing the insect cell transduced in step (b) in a serum-free medium to produce a coronavirus virus-like particle;
wherein the coronavirus virus-like particle comprises a coronavirus spike(S) protein.

2. The method of claim 1, wherein the insect cell is a mosquito cell.

3. The method of claim 2, wherein the mosquito cell is selected from a C6/36 cell or an AP-61 cell.

4. The method of claim 1, wherein the coronavirus spike protein gene is derived from β-coronaviruses (severe acute respiratory syndrome coronavirus (SARS-COV), Middle East respiratory syndrome coronavirus (MERS-COV), or severe acute respiratory syndrome coronavirus 2 (SARS-COV-2)).

5. The method of claim 1, wherein the coronavirus spike protein gene is a prefusion-stabilized full-length spike protein gene and comprises a mutation at the furin cleavage site and a 2P (two-proline) mutation.

6. The method of claim 1, wherein the recombinant baculovirus further comprises:
an hr1 pag1 mosquito promoter;
a Japanese encephalitis virus prM signal peptide;
a RhPV 5′-UTR internal ribosome entry site (IRES);
an enhanced green fluorescent protein (eGFP) gene; and
a translation stop codon.

7. The method of claim 1, wherein the serum-free medium comprises:
RPMI-1640 medium;
tryptose phosphate broth (TPB);
Pluronic® F-68;
peptone primatone;
yeastolate; and
a lipid mixture.

8. The method of claim 7, wherein the concentrations of the components in the serum-free medium are:
0.3% tryptose phosphate broth;
0.2% Pluronic® F-68;
0.5% peptone primatone;
0.4% yeastolate; and
0.1% lipid mixture.

9. A vaccine comprising the coronavirus virus-like particles prepared according to the method of claim 1.

10. The vaccine according to claim 9, wherein the vaccine is administered intranasally or by intramuscular injection.
